(19) United States
(12) Patent Application Publication
(10) Pub. No.: US 2024/0336948 A1
(43) Pub. Date: Oct. 10, 2024

(54) METHOD FOR PRODUCING A RECOMBINANT BACTERIAL COLLAGEN-LIKE PROTEIN (CLP)

(71) Applicant: Evonik Operations GmbH, Essen (DE)

(72) Inventors: Christian BANGERT, Linsengericht (DE); Edgar Propenauer, Waechtersbach (DE)

(73) Assignee: Evonik Operations GmbH, Essen (DE)

(21) Appl. No.: 18/682,010

(22) PCT Filed: Aug. 3, 2022
(86) PCT No.: PCT/EP2022/071826

§ 371 Date: Feb. 7, 2024

(30) Foreign Application Priority Data
Aug. 9, 2021 (EP) 21190340.6

(51) Int. Cl. C12P 21/02; C07K 14/78

(52) U.S. Cl. CPC C12P 21/02; C07K 14/78

(57) ABSTRACT

A method is developed for producing a recombinant collagen-like protein (CLP). The method includes fermenting of a host cell, expressing a polynucleotide encoding an amino acid sequence encoding a CLP, incubating the fermentation broth for at least 1 hour at not more than 25° C. for folding of the CLP, and purifying of the CLP by solvent precipitation.

The present invention relates to a novel method for producing and purification of a recombinant collagen-like protein (CLP). More specifically, the present invention relates to the purification of triple helical Scl2 protein by precipitation using organic solvents.

Collagen-like proteins (CLPs) of bacterial origin (the most industrially relevant being the product of Streptococcus pyogenes) have considerably interesting mechanical properties, similar to those of higher eukaryotes' collagen proteins, without needing the complex maturing steps required for the eukaryotic counterparts. CLPs present a common structure: two alpha helixes, stabilizing each other, constitute a “V domain”, which is followed by a rod-like, structural collagen domain (CL). After the collagen domain, typically a membrane anchor (GPI-like) is present at the C-terminal end of the protein.

Expression of collagen-like proteins have been attempted in several systems, including Escherichia coli, Pichia pastoris and Saccharomyces cerevisiae (J. Biol. Chem. 277, 27312-27318).

For expression in E. coli the construct of choice for such production carries a specific and necessary modification, in order to efficiently remove the potentially immunogenic V domain: such modification consists of a protease cleavage site typically inserted between the V domain and the collagen sequence. Due to this modification, the protein produced by the bacterial host must be extracted from the intracellular fraction and processed with a specific protease to remove the V domain. The mature protein, consisting of only the collagen-like domain, must be purified against the cleaved V domain, the whole intracellular protein content and the protease added to process the immature CLP. Such workflow greatly hinders the cost-effectiveness of the whole process, due to 1) the product of choice must be separated from the whole content of expression host cells, and 2) proteases are typically expensive enzymes.

As described in various publications (Lukomski et al. 2002, Brodsky et al. 2009) the current understanding of folding of the Scl2 protein is that the V-domain is required for folding three Scl2 protein monomers into one triple helical structure in vitro (Lukomski et al. reveals in vivo folding without V-domain). Even though the V-domain might have a positive effect on this process it was found that it's not the sole factor for folding the protein. It could be shown that a proper folding also takes place in absence of the V-domain. The main factors identified are concentration of the Scl2 monomer, temperature, time, pH-Value und salt concentration.

Since the V-domain was thought to be crucial for production of triple helical Scl2 it was never considered to remove this sequence leading to the following challenges.
The V-domain makes up for approximately one third of the whole sequence and hinders the protein to be transported out of the Pichia pastoris host. This requires a complex downstream process containing cell lysis to remove the target protein from the cell.
The V-domain itself has pathogenic properties and needs to be removed during the purification process. This is done by a protease digest. Usage of a protease is quite costly, and it needs to be removed during downstream as well.

The following summary shows the process steps required for a product purification using a Scl2 construct with V-domain attached:
Cell separation (Centrifugation)
Cell lysis (Pressure homogenizer)
V-domain removal (Protease digest)
Removal of cell debris (pH-shift, centrifugation)
Purification (Solvent precipitation)
Washing (TFF)
Further purification (IEX)

Such a process is disclosed in Peng et al. (Appl. Microbiol. Biotechnol., 98:1807-1815, 2014) for example.

This invention describes a novel process to produce collagen-like proteins (CLPs) in several hosts.

In contrast to mammalian collagen, the collagen-like protein Scl2 has decreased solubility in the presence of water miscible organic solvents like acetone, ethanol and 2-propanol. For example, at 15% 2-propanol, 4 g/L triple helical Scl2 and 5° C. solubility is less than 10%. This property has never been revealed in literature while for mammalian collagen extraction protocols at up to 50% ethanol have been published.

The purification of triple helical Scl2 protein originating from a Pichia pastoris fermentation requires the removal of a quite complex matrix. The fermentation supernatant contains salts, carbohydrates, fat, proteases and other compounds. The first approach of removing these compounds was to perform a series of ultrafiltrations.

After cell separation (via centrifugation) and folding (via cooling of the concentrate) three rounds of ultrafiltration are performed. In the first ultrafiltration step the triple helical Scl2 protein is unfolded at 40° C. and filtered through a 100 kD membrane. This step serves to remove large sized impurities. The collected permeate is then concentrated in the consecutive 10 kD filtration. After folding the Scl2 protein a last ultrafiltration is performed to remove small sized impurities. This process can't provide Scl2 protein with a purity of >50 w %.

Therefore, the goal of the present invention was to provide a process for the production of a recombinant collagen-like protein with a high purity.

In this context it was found unexpectedly it is possible to purify collagen-like proteins with the help of solvent precipitation.

Therefore, the invention provides a novel method for producing a recombinant collagen-like protein (CLP) comprising the following steps:
a) fermentation of a host cell, expressing a polynucleotide encoding an amino acid sequence encoding a CLP,
b) incubating the fermentation broth for at least 1 h at not more than 25° C. for folding of the CLP,
c) purification of the CLP by solvent precipitation.

The host cell is preferably selected from bacterial, yeast of plant cells. It is preferred to use bacterial or yeast cells.

It is preferred, when the CLP has an amino acid sequence that is at least 60% identical of SEQ ID NO: 1, SEQ ID NO:2, SEQ ID NO:3, SEQ ID NO:4, SEQ ID NO:5, SEQ ID NO:6, SEQ ID NO:7, SEQ ID NO:8 or SEQ ID NO:9.

It is preferred, when the amino acid sequence comprises a deletion of between 38 and 90 amino acids at the N-terminus of the amino acid sequence of SEQ ID NO:1. This includes a complete deletion of the N-terminal V-domain (comprising 74 amino acids) and different truncations of the V-domain of at least 38 amino acids.

In a preferred embodiment, the amino acid sequence that is at least 60%, identical to the amino acid sequence of SEQ ID NO:2, SEQ ID NO:3, SEQ ID NO:4, SEQ ID NO:5, SEQ ID NO:6, SEQ ID NO: 7, SEQ ID NO:8 or SEQ ID NO:9.

In a preferred configuration the amino acid sequence is at least 90%, 92%, 94%, 96%, 97%, 98%, 99% or 100%, preferably 97%, particularly preferably 98%, very particularly preferably 99%, and extremely preferably 100%, identical to the amino acid sequence of SEQ ID NO:2, SEQ ID NO:3, SEQ ID NO:4, SEQ ID NO:5, SEQ ID NO:6, SEQ ID NO:7, SEQ ID NO:8 or SEQ ID NO:9.

In a preferred embodiment of the present invention the CLP is a bacterial collagen-like protein from Streptococcus pyogenes.

In a preferred embodiment, the method for producing a recombinant collagen-like protein (CLP) further comprises the following steps:
accumulation of the CLP in the medium, wherein a fermentation broth is obtained and separating the host cells from the fermentation broth, or
accumulation of the CLP in the host cell and extracting the CLP from the host cells (e.g. for E. coli).

In a preferred embodiment, the solvent precipitation in step c) is performed at a solvent concentration of at least 5%, preferably at least 10%, more preferably at least 15%.

In a preferred embodiment, solvent precipitation is performed at a temperature 37° C. or less, preferably 34° C. or less, more preferably 32° C. or less, most preferably 24° C. or less or 20° C. or less.

In a preferred embodiment, the solvent used for solvent precipitation is an organic solvent, preferably an organic polar solvent.

In a preferred embodiment, the solvent used for solvent precipitation is a polar solvent with a relative polarity of less than 0.9, more preferably less than 0.8, most preferably less than 0.7.

In a preferred embodiment, the solvent used for solvent precipitation is selected from 2-propanol, ethanol, acetone and dimethyl sulfoxide (DMSO).

It is preferred, when the method according to the present invention further comprises one or more of the following steps:
d) drying, preferably spray-drying, freeze-drying or contact drying at low temperatures below 37° C.;
e) one or more additional purification steps selected from: ultrafiltration, solvent precipitation, tangential flow filtration (TFF), ion exchange chromatography;
f) incubation with a protease, preferably trypsin, pepsin, chymosin, for cleaving of the N-terminal variable globular (V) domain.

In a preferred embodiment, folding of CLP in step b) is performed
at a temperature between-80° C. and 25° C., preferably between 0° C. and 20° C.,
for a time between 1 h and 48 h, preferably between 1 h and 24 h,
with a CLP-concentration of at least 1 mg/ml, preferably at least 4 mg/ml.

In a preferred embodiment, the nucleotide sequence is a replicable nucleotide sequence encoding the collagen-like protein from Streptococcus pyogenes.

In a preferred embodiment, the host cell is a yeast cell, preferably Pichia pastoris or a bacterial cell, preferably E. coli, Corynebacterium or Brevibacterium.

According to the present invention, the microorganism used for the fermentation is characterized in that the nucleotide sequence according to the invention is integrated in a chromosome. Homologous recombination permits, with use of the vectors according to the invention, the exchange of DNA sections on the chromosome for polynucleotides according to the invention which are transported into the cell by the vector. For efficient recombination between the ring-type DNA molecule of the vector and the target DNA on the chromosome, the DNA region that is to be exchanged containing the polynucleotide according to the invention is provided at the ends with nucleotide sequences homologous to the target site; these determine the site of integration of the vector and of exchange of the DNA.

The microorganism may be a microorganism in which the nucleotide sequence is present in overexpressed form.

The microorganism may be characterized in that the microorganism has the capability of producing and secreting a fine chemical. The fine chemical being preferably a collagen-like protein.

Overexpression is taken to mean, generally, an increase in the intracellular concentration or activity of a ribonucleic acid, a protein (polypeptide) or an enzyme, compared with the starting strain (parent strain) or wild-type strain, if this is the starting strain. A starting strain (parent strain) is taken to mean the strain on which the measure leading to the overexpression was carried out.

In the overexpression, the methods of recombinant overexpression are preferred. These include all methods in which a microorganism is produced using a DNA molecule provided in vitro. Such DNA molecules comprise, for example, promoters, expression cassettes, genes, alleles, encoding regions etc. These are converted into the desired microorganism by methods of transformation, conjugation, transduction or like methods.

The extent of the expression or overexpression can be established by measuring the amount of the mRNA transcribed by the gene, by determining the amount of the polypeptide, and by determining the enzyme activity.

The culture medium or fermentation medium that is to be used must appropriately satisfy the demands of the respective strains. Descriptions of culture media of various microorganisms are contained in the handbook “Manual of Methods for General Bacteriology” of the American Society for Bacteriology (Washington D.C., USA, 1981). The terms culture medium and fermentation medium or medium are mutually exchangeable.

As carbon source, sugars and carbohydrates can be used, such as, e.g., glucose, sucrose, lactose, fructose, maltose, molasses, sucrose-containing solutions from beet sugar or sugar cane processing, starch, starch hydrolysate and cellulose, oils and fats, such as, for example, soybean oil, sunflower oil, groundnut oil and coconut fat, fatty acids, such as, for example, palmitic acid, stearic acid and linoleic acid, alcohols such as, for example, glycerol, methanol and ethanol, and organic acids, such as, for example, acetic acid or lactic acid.

As nitrogen source, organic nitrogen compounds such as peptones, yeast extract, meat extract, malt extract, corn-steep liquor, soybean meal and urea or inorganic compounds such as ammonium sulphate, ammonium chloride, ammonium phosphate, ammonium carbonate and ammonium nitrate can be used. The nitrogen sources can be used individually or as a mixture.

As phosphorus source, phosphoric acid, potassium dihydrogenphosphate or dipotassium hydrogenphosphate or the corresponding sodium-containing salts can be used.

The culture medium must, in addition, contain salts, for example in the form of chlorides or sulphates of metals such as, for example, sodium, potassium, magnesium, calcium and iron, such as, for example, magnesium sulphate or iron sulphate, which are necessary for growth. Finally, essential growth substances such as amino acids, for example homoserine and vitamins, for example thiamine, biotin or pantothenic acid, can be used in addition to the above-mentioned substances.

Said starting materials can be added to the culture in the form of a single batch or supplied in a suitable manner during the culturing.

Basic compounds such as sodium hydroxide, potassium hydroxide, ammonia or ammonia water, or acid compounds such as phosphoric acid or sulphuric acid, are used in a suitable manner for pH control of the culture. The pH is generally adjusted to 6.0 to 8.5, preferably 6.5 to 8. For control of foam development, antifoams can be used, such as, for example, polyglycol esters of fatty acids. For maintaining the stability of plasmids, suitable selectively acting substances such as, for example, antibiotics, can be added to the medium. The fermentation is preferably carried out under aerobic conditions. In order to maintain said aerobic conditions, oxygen or oxygen-containing gas mixtures such as, for example, air, are introduced into the culture. The use of liquids that are enriched with hydrogen peroxide is likewise possible. Optionally, the fermentation is carried out at superatmospheric pressure, for example at a superatmospheric pressure of 0.03 to 0.2 MPa. The temperature of the culture is usually 20° C. to 45° C., and preferably 25° C. to 40° C., particularly preferably 30° C. to 37° C. In the case of batch or fed-batch processes, the culturing is preferably continued until an amount sufficient for the measure of obtaining the desired organic chemical compound has formed. This goal is usually reached within 10 hours to 160 hours. In continuous processes, longer culture times are possible. Owing to the activity of the microorganisms, enrichment (accumulation) of the fine chemicals in the fermentation medium and/or in the cells of the microorganisms occurs.

Examples of suitable fermentation media may be found, inter alia, in patent documents U.S. Pat. Nos. 5,770,409, 5,990,350, 5,275,940, WO 2007/012078, U.S. Pat. No. 5,827,698, WO 2009/043803, U.S. Pat. No. 5,756,345 or U.S. Pat. No. 7,138,266; appropriate modifications may optionally be carried out to the requirements of the strains used.

The process may be characterized in that it is a process which is selected from the group consisting of batch process, fed-batch process, repetitive fed-batch process and continuous process.

The process may be further characterized in that the fine chemical or a liquid or solid fine chemical-containing product is obtained from the fine chemical-containing fermentation broth.

The performance of the processes or fermentation processes according to the invention with respect to one or more of the parameters selected from the group of concentration (compound formed per volume), yield (compound formed per carbon source consumed), volumetric productivity (compound formed per volume and time) and biomass-specific productivity (compound formed per cell dry mass or bio dry mass and time or compound formed per cell protein and time) or other process parameters and combinations thereof, is increased by at least 0.5%, at least 1%, at least 1.5% or at least 2%, based on processes or fermentation processes with microorganisms in which the promoter variant according to the invention is present.

Owing to the measures of the fermentation, a fermentation broth is obtained which contains the desired fine chemical, preferably amino acid or organic acid.

Then, a product in liquid or solid form that contains the fine chemical is provided or produced or obtained.

A fermentation broth is taken to mean, in a preferred embodiment, a fermentation medium or nutrient medium in which a microorganism was cultured for a certain time and at a certain temperature. The fermentation medium, or the media used during the fermentation, contains/contain all substances or components that ensure production of the desired compound and typically ensure growth and/or viability.

On completion of the fermentation, the resultant fermentation broth accordingly contains
a) the biomass (cell mass) of the microorganism resulting from growth of the cells of the microorganism,
b) the desired fine chemical formed in the course of the fermentation,
c) the organic by-products possibly formed in the course of the fermentation, and
d) the components of the fermentation medium used, or of the starting materials, that are not consumed by the fermentation, such as, for example, vitamins such as biotin, or salts such as magnesium sulphate.

The organic by-products include substances which are generated in addition to the respective desired compound by the microorganisms used in the fermentation and are possibly secreted.

The fermentation broth is withdrawn from the culture vessel or the fermentation container, optionally collected, and used for providing a product in liquid or solid form containing the fine chemical. The expression “obtaining the fine chemical-containing product” is also used therefor. In the simplest case, the fine chemical-containing fermentation broth withdrawn from the fermentation container is itself the product obtained.

By way of one or more of the measures selected from the group
a) partial (>0% to <80%) to complete (100%) or virtually complete (≥80%, ≥90%, ≥95%, ≥ 96%, ≥97%, ≥98%, ≥99%) removal of the water,
b) partial (>0% to <80%) to complete (100%) or virtually complete (≥80%, ≥90%, ≥95%, ≥ 96%, ≥97%, ≥98%, ≥99%) removal of the biomass, wherein this is optionally inactivated before the removal,
c) partial (>0% to <80%) to complete (100%) or virtually complete (≥80%, ≥90%, ≥95%, ≥ 96%, ≥97%, ≥98%, ≥99%, ≥99.3%, ≥99.7%) removal of the organic by-products formed in the course of the fermentation, and
d) partial (>0%) to complete (100%) or virtually complete (≥80%, ≥90%, ≥95%, ≥96%, ≥ 97%, ≥98%, ≥99%, ≥99.3%, ≥99.7%) removal of the components of the fermentation medium used or the starting materials that are not consumed by the fermentation,
a concentration or purification of the desired organic chemical compound is achieved from the fermentation broth. In this manner, products are isolated that have a desired content of the compound.

The partial (>0% to <80%) to complete (100%) or virtually complete (≥80% to <100%) removal of the water (measure a)) is also termed drying.

In a variant of the process, by complete or virtually complete removal of the water, the biomass, the organic by-products and the non-consumed components of the fermentation medium used, pure (≥ 80% by weight, ≥90% by weight) or high-purity (≥95% by weight, ≥97% by weight, ≥99% by weight) product forms of the desired organic chemical compound, preferably collagen-like protein, are successfully arrived at. For the measures according to a), b), c) or d), a great variety of technical instructions are available in the prior art.

In the case of processes for producing collagen-like protein processes are preferred in which products are obtained that do not contain any components of the fermentation broth. These products are used, in particular, in human medicine, in the pharmaceuticals industry, and in the food industry.

### EXAMPLES

The collagen-like protein was produced in the yeast host cell Pichia pastoris by fermentation. To produce Scl2 from Streptococcus pyogenes in Pichia pastoris, the sequence of the collagen-like protein (full-length protein and truncated variants and no-V-domain variant), has been codon optimized using different algorithms, and cloned in a secretion vector for Pichia pastoris. The sequences used are summarized in SEQ ID NO:1 to SEQ ID NO:9. For each of the specific sequences, a vector was transformed in Pichia pastoris following standard protocol and a standard expression protocol in fed-batch mode was applied (Damasceno, L. M., Huang, C J. & Batt, C. A. Protein secretion in Pichia pastoris and advances in protein production. Appl Microbiol Biotechnol 93, 31-39 (2012)). The collagen domain of the Scl2p protein was detected via HPLC analysis in the supernatant of cell culture. Upon fermentation, supernatant has been separated from biomass via centrifugation (12000 g, 5 mins at room temperature).

The collagen domain of the Scl2p protein based on the sequences SEQ ID NO:1 to SEQ ID NO:9 could be produced under similar conditions using either E. coli, B. choshinensis or C. glutamicum. In case of a production in yeast or C. glutamicum, the collagen domain is secreted by the cell. No cell lysis is needed as an initial purification step in this approach. In case of a production in E. coli a cell lysis is mandatory to remove the collagen domain from the cell.

The full-length collagen-like protein, a truncated variant (truncation 3) and the no-V-domain variant (based on the gene scl2 from Streptococcus pyogenes) were also expressed in Brevibacillus choshinensis. Therefore, the corresponding DNA sequences were cloned into a suitable secretion vector for B. choshinensis. Transformation of B. choshinensis with the new constructed plasmids was done according to Mizukami et al. 2010 (Curr Pharm Biotechnol 2010, 13:151-258).

The B. choshinensis strains were analyzed for their ability to produce the different collagen proteins in batch cultivations at 33° C. and pH 7 using the DASGIP® parallel bioreactor system from Eppendorf (Hamburg, Germany). The fermentation was performed using 1 L reactors. The production medium (TM medium, Biomed Res Int 2017, 2017:5479762) contained 10 g/L glucose. Upon fermentation, supernatant has been separated from biomass by centrifugation and was used for SDS PAGE analysis. For all three variants, collagen-like protein was produced.

The full-length collagen-like protein and the no-V-domain variant (based on the gene scl2 from Streptococcus pyogenes) were also expressed in Corynebacterium glutamicum. Therefore, the corresponding DNA sequences were cloned together with an upstream located signal peptide for protein secretion into a shuttle vector for C. glutamicum (Biotechnology Techniques 1999, 13:437-441.). The C. glutamicum strain ATCC 13032 was transformed with the new constructed plasmids by means of electroporation as described by Ruan et al. (Biotechnology Letters 2015, 37:2445-2452).

The C. glutamicum strains were analyzed for their ability to produce the different collagen proteins in fed-batch cultivations at 30° C. and pH 7 using the DASGIP® parallel bioreactor system from Eppendorf (Hamburg, Germany). The fermentation was performed using 1 L reactors. The production medium contained 20 g/L glucose in the batch phase and the fed-batch phase was run with a glucose feed of 4 g/L*h. Upon fermentation, supernatant has been separated from biomass by centrifugation and was used for HPLC analysis. For both variants, collagen protein was produced. For the truncated variant of the collagen-like protein, product titer was higher as for the full-length variant.

The process steps are summarized below:
1. Production of collagen-like protein in yeast, E. coli or Corynebacterium
2. Cell lysis (only for E. coli)
3. Cell separation (Filtration or centrifugation)
4. Folding of the CL single strand to form a triple helical structure
5. Further purification by solvent precipitation and ultrafiltration
6. Freeze dry of the purified CL protein

### 1. Precipitation of the Collagen-Like Protein Using Different Solvents

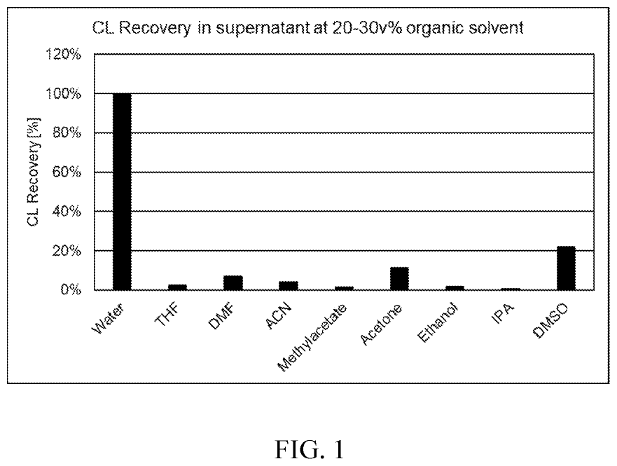

The solubility of the collagen-like protein was analyzed in various organic solvents. Therefor freeze-dried collagen domain of the Scl2p protein coming from a production in the yeast Pichia pastoris is dissolved at a concentration of 5 g/L in DI water. 800 μL of this solution is placed in 1.5 ml Eppendorf cups at a temperature of 5° C. 200 μL of pre-cooled MilliQ water (comparison), DMF, Acetonitrile, THF, methyl acetate, Acetone, Ethanol, Isopropanol (IPA) or DMSO are added to the test samples. The mixtures are then incubated in a Thermomixer for 15 min at 5° C. and 1000 rpm. The mixture is centrifuged for 3 min (16100×g/5° C.) and the supernatant injected after dilution with 50 mM Na-Phosphate buffer pH7.2 by factor 10. The CL recovery in organic solvents is summarized in FIG. 1.

### 2. Precipitation at Various Solvent Concentrations

In addition to that the solubility of collagen domain of the Scl2p protein in Isopropanol (IPA) and Acetone is determined in a solvent concentration range of 5-40 v % and in a temperature range of 4-30° C.

Freeze dried collagen domain of the Scl2p protein coming from a production in bacteria (C. glutamicum or B. choshinensis) is dissolved at a concentration of 6 g/L in 50 mM Na-Phosphate buffer pH7.2. 800 μL CL stock solution are placed in 1.5 mL Eppendorf cups at a temperature of 5° C. 200 μL of pre-cooled MilliQ water (comparison), IPA or Acetone at varying amounts are added to the test samples. The mixtures are then incubated in a Thermomixer for 15 min at 5° C. and 1000 rpm.

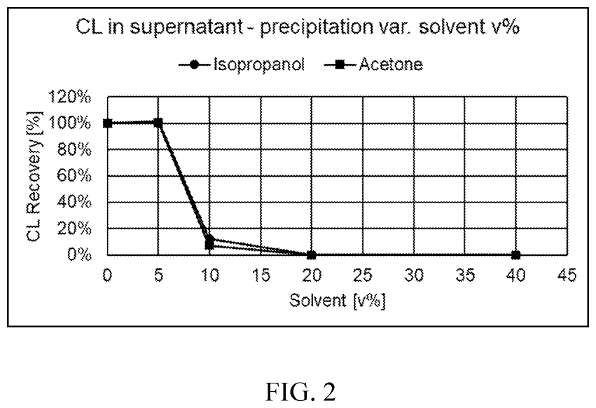

The mixture is centrifuged for 3 min (16100×g/5° C.) and the supernatant injected on SEC after dilution with 50 mM Na-Phosphate buffer pH7.2. The results are summarized in FIG. 2.

### 3. Precipitation at Various Temperatures

Freeze dried collagen domain of the Scl2p protein coming from a production in bacteria (C. glutamicum or B. choshinensis) is dissolved at a concentration of 6 g/L in 50 mM Na-Phosphate buffer pH7.2. 800 μL CL stock solution are placed in 1.5 mL Eppendorf cups at varying temperatures. 200 μL of pre-cooled MilliQ water (comparison), IPA or Acetone are added to the test samples. The mixtures are then incubated in a Thermomixer for 15 min at varying temperatures and 1000 rpm.

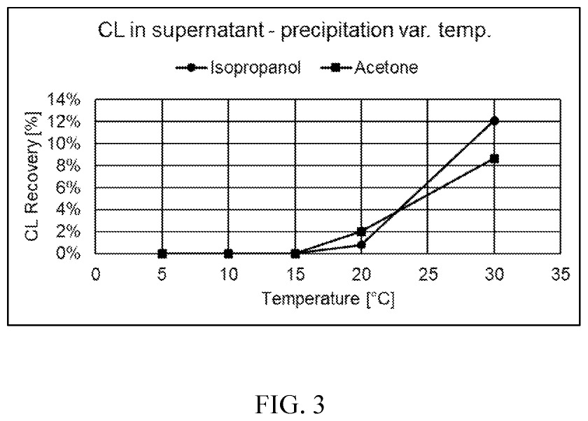

The mixture is centrifuged for 3 min (16100×g/5° C.) and the supernatant injected on SEC after dilution with 50 mM Na-Phosphate buffer pH7.2. The results are summarized in FIG. 3.

### 4. Purification of the Collagen-Like Protein

After cell separation (via centrifugation) and folding of the collagen domain of the Scl2p protein (via cooling of the concentrate) it was purified using precipitation with 2-Propanol at 15 v %. After precipitation of the collagen domain of the Scl2p protein a centrifugation was performed. The pellet was dissolved in water, the triple helical Scl2 protein was unfolded at 40° C. and filtered through a 100 kD membrane. This step serves to remove large sized impurities. The collected permeate was then concentrated in the consecutive 10 kD filtration. The retentate was washed to remove small sized impurities.

By that means a triple helical Scl2 protein purity>75 w % was achieved.

### Protein Sequences

SEQ ID NO:1 Streptococcus pyogenes Collagen-like protein (CLP), full length protein
SEQ ID NO:2 Streptococcus pyogenes CLP, truncation 3
SEQ ID NO:3 Streptococcus pyogenes CLP, truncation 5
SEQ ID NO:4 Streptococcus pyogenes CLP, no V-domain
SEQ ID NO:5 Streptococcus pyogenes CLP, truncation 5 (AGPR mutant)
SEQ ID NO:6 Streptococcus pyogenes CLP, truncation 5 (QGPR mutant)
SEQ ID NO:7 Streptococcus pyogenes CLP, truncation 5 (VGPA mutant)
SEQ ID NO:8 Streptococcus pyogenes CLP, truncation 5 (SGPR mutant)
SEQ ID NO:9 Streptococcus pyogenes CLP, truncation 5 (VGPK mutant)

1. A method for producing a recombinant collagen-like protein (CLP) comprising:
a) fermenting a host cell, expressing a polynucleotide encoding an amino acid sequence encoding a CLP, forming a CLP,
b) incubating a fermentation broth for at least 1 h at not more than 25° C. for folding of the CLP,
c) purifying the CLP by solvent precipitation with a solvent.

2. The method according to claim 1, further comprising:
accumulating the CLP in a medium, wherein a fermentation broth is obtained and separating the host cell from the fermentation broth, or
accumulating the CLP in the host cell and extracting the CLP from the host cell.

3. The method according to claim 1, wherein the solvent precipitation in c) is performed at a solvent concentration of at least 5%.

4. The method according to claim 1, wherein the solvent precipitation is performed at a temperature of 37° C. or less.

5. The method according to claim 1, wherein the solvent used for the solvent precipitation is an organic solvent.

6. The method according to claim 1, wherein the solvent used for the solvent precipitation is a polar solvent with a relative polarity of less than 0.9.

7. The method according to claim 1, wherein the solvent used for the solvent precipitation is selected from the group consisting of 2-propanol, ethanol, acetone, dimethyl sulfoxide (DMSO), and mixtures thereof.

8. The method according to claim 1, further comprising:
d) drying of the CLP at low temperatures below 37° C.;
e) additionally purifying the CLP, wherein the one or more additional purifications are selected from the group consisting off ultrafiltration, solvent precipitation, tangential flow filtration (TFF), and ion exchange chromatography; and/or
f) incubating the CLP with a protease for cleaving an N-terminal variable globular (V) domain.

9. The method according to claim 1, wherein the folding of the CLP in b) is performed
at a temperature between −80° C. and 25° C.,
for a time between 1 h and 48 h,
with a CLP-concentration of at least 1 mg/ml.

10. The method according to claim 1, wherein the polynucleotide has a nucleotide sequence which is a replicable nucleotide sequence encoding the collagen-like protein from Streptococcus pyogenes.

11. The method according to claim 1, wherein the host cell is a yeast cell.

12. The method according to claim 1, wherein the solvent precipitation in c) is performed at a solvent concentration of at least 15%.

13. The method according to claim 1, wherein the solvent precipitation is performed at a temperature of 20° C. or less.

14. The method according to claim 1, wherein the solvent used for the solvent precipitation is an organic polar solvent.

15. The method according to claim 1, wherein the solvent used for the solvent precipitation is a polar solvent with a relative polarity of less than 0.7.

16. The method according to claim 8, wherein the drying in d) is spray-drying, freeze-drying, contact drying, or combinations thereof.

17. The method according to claim 8, wherein the protease in f) is trypsin, pepsin, chymosin, or mixtures thereof.

18. The method according to claim 9, wherein the folding of the CLP in b) is performed at a temperature between 0° C. and 20° C.

19. The method according to claim 9, wherein the folding of the CLP in b) is performed for a time between 1 h and 24 h.

20. The method according to claim 9, wherein the folding of the CLP in b) is performed with a CLP-concentration of at least 4 mg/ml.


SEQUENCE LISTING (9 sequences)

SEQ ID NO: 1 - protein, 330 aa, Streptococcus pyogenes
NHKVHMHHHHHHADEQEEKAKVRTELIQELAQGLGGIEKKNFPTLGDEDLDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 2 - protein, 292 aa, Streptococcus pyogenes
NHKVHMHHHHHHDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 3 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 4 - protein, 240 aa, Streptococcus pyogenes
GSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 5 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPAGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 6 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPQGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 7 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPAGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 8 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPSGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 9 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPKGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY